(19) United States
(12) Patent Application Publication
(10) Pub. No.: US 2021/0393211 A1
(43) Pub. Date: Dec. 23, 2021

(54) PERSONALIZED SLEEP CLASSIFYING METHODS AND SYSTEMS

(71) Applicant: Nox Medical ehf, Reykjavik (IS)

(72) Inventors: Sveinbjorn HOSKULDSSON, Reykjavik (IS); Jon Skirnir AGUSTSSON, Reykjavik (IS); Sigurdur Ægir JONSSON, Reykjavik (IS)

(21) Appl. No.: 17/351,933

(22) Filed: Jun. 18, 2021

Related U.S. Application Data
(60) Provisional application No. 63/041,013, filed on Jun. 18, 2020.

(51) Int. Cl. A61B 5/00; A61B 5/08; G16H 20/30; G16H 15/00; G06N 20/00

(52) U.S. Cl. CPC A61B 5/7267; A61B 5/0806; A61B 5/4806; G06N 20/00; G16H 20/30; G16H 15/00; A61B 5/4848

(57) ABSTRACT

Methods and systems are provided for creating a personalized sleep classifier for a subject. Sleep data are obtained from biosignals from a subject in a High-Accuracy Sleep Study (HASS). Sleep data are also obtained from biosignals from the subject in a Simplified Sleep Study (SSS), the High-Accuracy Sleep Study being obtained simultaneously from the subject with the Simplified Sleep Study. A high-resolution HASS sleep profile is developed from the sleep data of the High-Accuracy Sleep Study. A personalized sleep classifier is created that outputs a SSS sleep profile of the subject based on the sleep data from the Simplified Sleep Study. And the personalized sleep classifier is calibrated such the SSS sleep profile output by the personalized sleep classifier based on the Simplified Sleep Study of the subject approaches or aligns with the high-resolution HASS sleep profile based on the High-Accuracy Sleep Study of the subject.

### CROSS-REFERENCE TO RELATED APPLICATIONS

This application claims the benefit of and priority to U.S. Provisional Patent Application Ser. No. 63/041,013 filed on Jun. 18, 2020 and entitled “Personalized Sleep Classifying Method and System,” which application is expressly incorporated herein by reference in its entirety.

### FIELD OF THE DISCLOSURE

The present disclosure relates to systems, apparatuses, and methods for performing a personalized sleep study of a subject, and particularly for creating a personalized sleep classifier of a subject with high accuracy based on simplified sleep study signals obtained from the body of the subject.

### BACKGROUND

Measuring or studying sleep often includes classifying different states of sleep correctly and identifying arousals and other sleep events throughout the night. The Manual for Scoring Sleep and Associated Events 2.6 (2020) by the American Academy of Sleep Medicine (AASM) is helpful as it provides a description of the current state of the art in measuring and studying sleep, and is incorporated herein by reference in its entirety.

The result of sleep measuring or a sleep study often includes a Sleep Profile, which often includes a hypnogram and indexes related to different events detected. The hypnogram may include a chart representing different stages of sleep of a subject Sleep Profiles including hypnograms and sleep indexes have great clinical value for identifying sleep health and sleep disorders, and for determining the efficiency of treatment.

A hypnogram may depict how sleep stages evolve throughout the night where the patient's sleep alternates between the sleep stages of Wake, Rapid Eye Movement (REM) sleep, and the Non-REM sleep stages N1, N2, and N3. The Non-REM sleep stages represent an increasing sleep depth from light sleep being N1, N2 being a deeper sleep stage, and N3 representing deep sleep.

The sleep indexes of a sleep profile often include an expansive collection of indices derived from the sleep study. These indices include, but are not limited to an Arousal Index, Apnea-Hypopnea Index, Oxygen Desaturation Index, Limb Movement Index, Periodic Limb Movement Index, Total Sleep Time, Wake After Sleep Onset, and Position, which will be described more fully herein.

A Standard High-Accuracy Sleep Study (HASS) for clinical purposes may be referred to as polysomnography (PSG). The setup for a PSG is often complicated and requires professional assistance to accurately setup and perform. So a significant drawback to PSG is that PSG cannot be practically deployed for multiple nights, such as for studying and trending patients Sleep Profiles during treatment.

Other forms of High-Accuracy Sleep Study (HASS) also exist. These forms of the HASS record physiological signals which are sufficient to accurately detect sleep events such as sleep stages, arousals, or other events relevant to the medical condition of interest. These alternative forms of the HASS may vary depending on the medical condition of interest.

Simplified Sleep Studies (SSS), which may include a subset of the signals of PSG or an alternative set of signals and are used to detect sleep events such as sleep stages, arousals, apneas, hypopneas, desaturation, arrythmias or any other events or combination thereof. Simplified Sleep Studies (SSS) can be applied by the patient himself and be repeated for multiple nights. But a significant problem with current Simplified Sleep Studies (SSS) is that they are generally not considered to be sufficiently accurate to detect sleep events such as sleep stages, arousals, or other events as defined by the American Academy of Sleep Medicine (AASM).

### SUMMARY

A method for creating a personalized sleep classifier for a subject is provided. The method comprises: obtaining sleep data from biosignals received from the subject in a High-Accuracy Sleep Study (HASS) and sleep data from biosignals received from the subject in a Simplified Sleep Study (SSS), the data from the High-Accuracy Sleep Study (HASS) being obtained from the subject during a period of time that is simultaneous with the period during which the data from the Simplified Sleep Study (SSS) is obtained from the subject; developing a high-resolution HASS sleep profile from the sleep data of the High-Accuracy Sleep Study (HASS); creating a personalized sleep classifier that outputs a SSS sleep profile of the subject based on the sleep data from the Simplified Sleep Study (SSS); and calibrating the personalized sleep classifier such the SSS sleep profile output by the personalized sleep classifier based on the Simplified Sleep Study (SSS) of the subject approaches, aligns with, or fits the high-resolution HASS sleep profile based on the High-Accuracy Sleep Study (HASS) of the subject.

Further, a method for creating a personalized sleep classifier for one or more subjects of a focused group of subjects. This method comprises: obtaining sleep data from biosignals received from the focused group of subjects in a High-Accuracy Sleep Study (HASS) and sleep data from biosignals received from the focused group of subjects in a Simplified Sleep Study (SSS), the data from the High-Accuracy Sleep Study (HASS) being obtained from the focused group of subjects during a period of time that is simultaneous with the period during which the data from the Simplified Sleep Study (SSS) is obtained from the focused group of subjects; developing a high-resolution HASS sleep profile from the sleep data of the High-Accuracy Sleep Study (HASS); creating a personalized sleep classifier that outputs a SSS sleep profile of the focused group of subjects based on the sleep data from the Simplified Sleep Study (SSS); and calibrating the personalized sleep classifier for one or more of the focused group of subjects such that the SSS sleep profile output by the personalized sleep classifier based on the Simplified Sleep Study (SSS) of the one or more of the focused group of subjects approaches, aligns with, or fits the high-resolution HASS sleep profile based on the High-Accuracy Sleep Study (HASS) one or more of the focused group of subjects, wherein the focused group of subjects share one or more same characteristics, including age, sex, diagnosed clinical condition, weight, race/ethnicity, BMI, treatment with a same medication, or health condition.

A computing system is provided for creating a personalized sleep classifier, the computing system comprising: one or more processors; one or more computer-readable storage devices having stored thereon computer-executable instructions that are structured such that, when executed by the one or more processors, cause the computing system to perform the following: obtain sleep data from biosignals received from the subject of a High-Accuracy Sleep Study (HASS) and sleep data from biosignals received from the subject in a Simplified Sleep Study (SSS), the data from the High-Accuracy Sleep Study (HASS) being obtained from the subject during a period of time that is simultaneous with the period during which the data from the Simplified Sleep Study (SSS) is obtained from the subject; develop a high-resolution HASS sleep profile from the sleep data of the High-Accuracy Sleep Study (HASS); create a personalized sleep classifier that outputs a SSS sleep profile of the subject based on the sleep data from the Simplified Sleep Study (SSS); calibrate the personalized sleep classifier such the SSS sleep profile output by the personalized sleep classifier based on the Simplified Sleep Study (SSS) of the subject approaches, aligns with, or fits the high-resolution HASS sleep profile based on the High-Accuracy Sleep Study (HASS) of the subject.

A hardware storage device is provided having stored thereon computer executable instructions which, when executed by one or more processors of a computer system, configure the computer system to perform a method comprising: obtaining sleep data from biosignals received from the subject in a High-Accuracy Sleep Study (HASS) and sleep data from biosignals received from the subject in a Simplified Sleep Study (SSS), the data from the High-Accuracy Sleep Study (HASS) being obtained from the subject during a period of time that is simultaneous with the period during which the data from the Simplified Sleep Study (SSS) is obtained from the subject; developing a high-resolution HASS sleep profile from the sleep data of the High-Accuracy Sleep Study (HASS); creating a personalized sleep classifier that outputs a SSS sleep profile of the subject based on the sleep data from the Simplified Sleep Study (SSS); and calibrating the personalized sleep classifier such the SSS sleep profile output by the personalized sleep classifier based on the Simplified Sleep Study (SSS) of the subject approaches, aligns with, or fits the high-resolution HASS sleep profile based on the High-Accuracy Sleep Study (HASS) of the subject.

### BRIEF DESCRIPTION OF THE DRAWINGS

FIG. 1 shows a PolySomnoGraphy (PSG) setup.

FIG. 2A and FIG. 2B each show screenshots from data recorded during a PSG sleep study.

FIG. 3 shows an embodiment of a practical High-Accuracy Sleep Study (HASS) setup.

FIG. 4A shows a schematic embodiment of a Self Applied Somnography (SAS) sleep study.

FIG. 4B shows a schematic embodiment of kit of devices to be used in a Self Applied Somnography (SAS) sleep study.

FIG. 5 shows an example of a calibration of a Simple Sleep Study (SSS) personalized sleep classifier.

FIG. 6 shows an embodiment of a method for creating a personalized sleep classifier.

FIG. 7 shows a schematic of a computer system that creates a personalized sleep classifier.

### DETAILED DESCRIPTION OF VARIOUS EMBODIMENTS

An object of the present application is to provide methods, systems and apparatuses that increase the accuracy and performance Simplified Sleep Studies (SSS) such that Simplified Sleep Studies (SSS) may serve as valuable tools for monitoring sleep or care management of an individual being treated for different medical disorders, and particularly to provide methods, systems and apparatuses with increased the accuracy and performance Simplified Sleep Studies (SSS) using devices applied by the individual patient himself without requiring nightly or regular professional assistance.

As noted above, the sleep indexes of a hypnogram often include an expansive collection of indices derived from the sleep study. These indices include, but are not limited to:
Arousal Index: The number of arousals per hour of sleep.
Apnea-Hypopnea Index: The number of complete breathing cessations (apneas), and severely restricted breathing (hypopnea) events per hour of sleep.
Oxygen Desaturation Index: The number of blood oxygen desaturation events per hour of sleep.
Limb Movement Index: The number of limb movement events per hour of sleep.
Periodic Limb Movement Index: The number of periodic limb movement events per hour of sleep.
Total Sleep Time: The total time duration of sleep during the sleep study.
Wake After Sleep Onset: The total time duration of wake periods from the first moment of sleep to the last moment of sleep.
Position: A measurement of the periods a patient is sleeping in a supine, prone, left-side, or right-side positions.

The accuracy of a sleep study may be considered to extend along a continuous spectrum. On one end of a sleep study is found what may be referred to as a Standard High-Accuracy Sleep Study (HASS) for clinical purposes may be called a polysomnography (PSG). As noted above, the setup for a PSG is complicated and requires professional assistance by certified or credential technologists to setup, perform, and monitor the PSG. PSG includes simultaneous recording of multiple signals, such as Electroencephalography (EEG), Electrooculography (EOG), Electromyography (EMG), Electrocardiography (ECG), Respiratory Flow, Respiratory Effort, Oximetry, Body Position, and/or more to achieve the required accuracy.

A significant drawback to PSG is that it is impractical to setup and monitor a PSG for multiple nights, such as for studying and trending patients Sleep Profiles during treatment.

Other forms of High-Accuracy Sleep Study (HASS) may exist. These forms of the HASS record physiological signals which are substantially equivalent to a PSG or are sufficient to accurately detect sleep events such as sleep stages, arousals, or other events as defined by the AASM. Forms of HASS may vary. For example, a HASS intended to correctly classify sleep stages must record EEG, EOG, EMG, and other physiological signals defined by the AASM. A HASS intended to correctly detect cortical arousals must record EEG and other physiological signals defined by the AASM. Furthermore, a HASS which is intended to measure events which occur during sleep must also measure the signals required to classify sleep stages. Alternative forms of a HASS my include, but are not limited to, a sleep study with EEG, EOG, and EMG montages which differ from the PSG defined montage.

Towards the other end of what may be considered the sleep study accuracy spectrum may be found what may be referred to as Simplified Sleep Studies (SSS). Simplified Sleep Studies (SSS) may be considered those sleep studies that use or reply on only a subset of the signals of PSG or an alternative set of signals. Simplified Sleep Studies (SSS) may be used to detect sleep events, including but not limited to, sleep stages, arousals, apneas, hypopneas, desaturation, arrythmias or any combination thereof. A significant advantage to Simplified Sleep Studies (SSS) is that the sensor devices required for the study can be applied by the patient himself or by untrained assistants, such a relatives or those that live with the patient. And Simplified Sleep Studies (SSS) may be repeated for multiple nights. A subset of signals for SSS may include, for example, any or any combination of Photo-PlethismoGraphy (PPG), Blood Oxygen Saturation, Activity, Temperature, Peripheral Arterial Tone (PAT), Respiratory flow, Respiratory Movements, limited EEG, EOG, ECG and EMG setup or other signal affected during sleep.

Although Simplified Sleep Studies (SSS) lack the information required to deliver a sleep profile of the same performance or accuracy as HASS, if the performance of Simplified Sleep Studies (SSS) could be increased, SSS could serve as valuable tools for monitoring sleep or care management of individuals being treated for different medical disorders.

Lastly, the terms or phrases relating to sleep studies, such as Simplified Sleep Study (SSS), High-Accuracy Sleep Study (HASS), and polysomnography (PSG), do not necessarily require that the sleep data obtained in such a sleep study be from a certain length of time, for example, from an entire night or multiple nights. Rather, as used herein, the sleep study, such as SSS, HASS, and PSG can be understood to mean sleep data for a period of time, which may be a full night sleep, but may also include a subset or combination of subsets of time (a specified number of minutes or hours), whether consecutive or not, within a night of sleep, or during a 24-hour day, week, or month, or longer. So a SSS or a HASS could be understood to mean sleep data obtained during less than an entire night of sleep, and multiple Simplified Sleep Studies (SSS), High-Accuracy Sleep Studies (HASS) may be performed during a single night or 24-hour period.

One of the issues with measuring sleep, is the complication of the hookup of the sleep study, particularly the electroencephalogram (EEG) used to determine sleep stages. Measuring sufficient sleep data for clinical use, requires a complicated hookup of sensors and simultaneous measurement of multiple, different physiological signals. The standard sleep study is called PolySomnoGraphy (PSG) for this very reason that multiple parameters are necessary to build an accurate picture of the subjects sleep and capture the effects of sleep disorders. Placing all the necessary sensors for a valid PSG study on the patient is significant work and requires professional assistance. This makes PSG costly and multi-night PSG studies impractical.

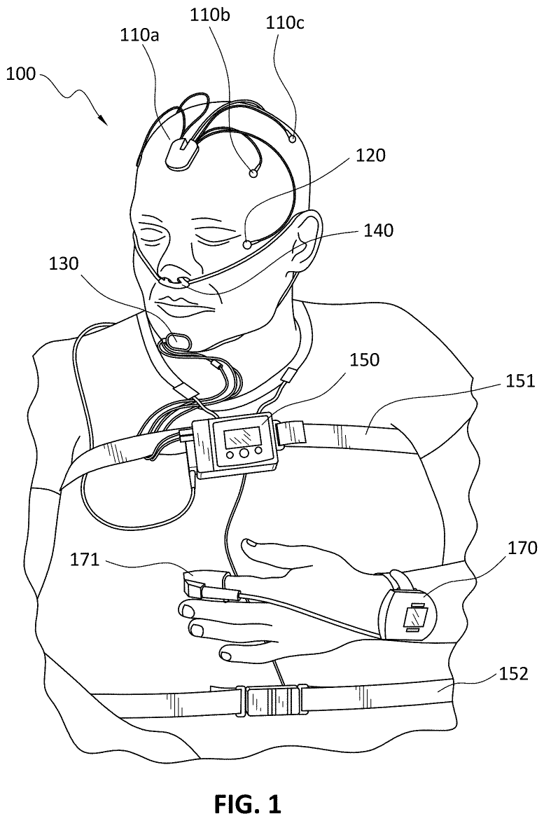

FIG. 1 shows a subject 100 and required devices and sensors of a typical PSG sleep study setup. FIG. 1 shows the subject or patient undergoing a PSG sleep study having EEG electrodes 110a, 110b, 110c fixed to his scalp. The EEG electrodes are placed on the forehead, the top of the head, at the back of the head, and behind the ears (not shown). The patient has EOG electrodes 120 placed next to his eyes and EMG electrodes 130 on the chin. Furthermore, the patient 100 has a nasal cannula 140 used to measure nasal breathing. The subject 100 also has respiratory inductance plethysmography (RIP) belts 151, 152 around his chest and abdomen, respectively, to measure breathing movements. Respiratory Inductive Plethysmography (RIP) uses the respiratory bands or belts 151, 152 to measure respiratory effort related areal changes. RIP technology includes a measurement of an inductance of a conductive belt or belts that encircles a respiratory region of a subject. Signals obtained from the RIP belts are obtained and recorded or processed by processor 150. The subject 100 also has a pulse oximeter 170 on the wrist and a corresponding sensor 171 on index finger measuring the blood oxygen saturation and pulse in the finger. Furthermore, the patient has an electro cardiogram (ECG), and leg EMG leads although not shown.

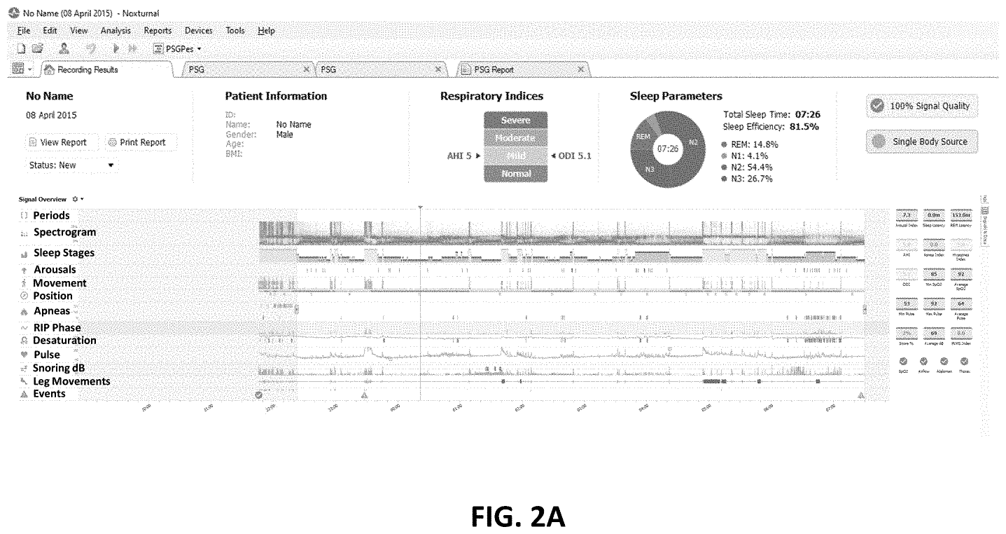

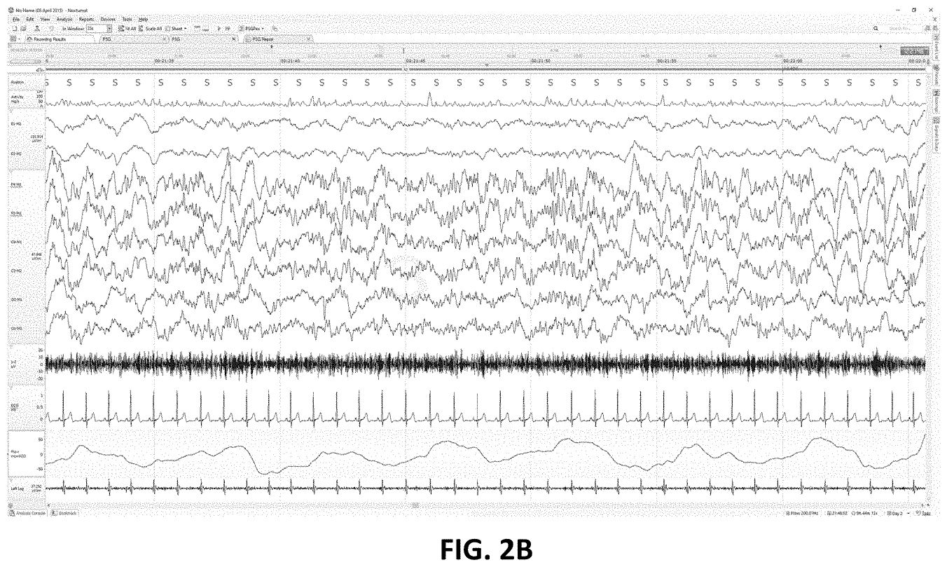

The PSG sleep study records around 1 Gb of data which is meticulously analyzed and scored by specially trained human experts, sleep technologists. The PSG sleep study is comprised of a multitude of physiological signal recorded throughout the duration of the night. FIGS. 2A and 2B show two screenshots from the data recorded during a sleep study.

FIG. 2A shows a screenshot from an overview page of a whole night sleep study and shows the results of the whole night sleep study along with the results of the manual analysis of the data. This includes the sleep stages, position of the patient's, respiratory events, blood oxygen saturation, pulse, snoring, leg movements, and much more. FIG. 2B shows a screenshot from the data used to determine the sleep stages of the patient. FIG. 2B shows a 30 second period from the sleep study typically used to score sleep stages.

Multiple attempts have been made to create Simplified Sleep Studies (SSS) where the patient can perform the hookup himself. Simplified Sleep Studies (SSS) have been developed both as medical devices and as commercial devices made available to consumers.

In the SSS medical devices the simplification has mostly been done by simplifying the PSG sleep study by recording a subset of the PSG signals. Reducing the number of signals has however a limiting effect on the outcome. The performance and accuracy of the SSS sleep profile is reduced. SSS methods generally only work on a limited group of subjects and the accuracy and performance is especially reduced when used by those having sleep disorders. For clinical purposes, SSS are however often used for specific sleep disorders, such as to confirm sleep apnea, where reasonable accuracy can be achieved based on the measure of respiratory and oximetry parameters only, allowing the EEG, EOG, ECG and EMG signals to be skipped. Further reduced sleep studies, such as those based on oximetry only, are also practiced with further reduced accuracy and mostly used for screening purposes only.

For commercial devices, which are not intended for clinical purposes, many types of SSS are available. Smart-Watches frequently deliver activity-graph and photo-plethysmography (PPG) signals that can be used to predict a limited sleep profile. The activity signal can, for example, be used to predict wake/sleep periods, where the PPG can be used to measure pulse and pulse rate variability that are known in healthy subjects to vary between stages of sleep, such as between rapid-eye-movement (REM) sleep and non-rapid-eye-movement (NREM) sleep. Long periods of no activity during NREM sleep frequently means that the patient's sleep is deep, whereas the sleep is more shallow if it is frequently interrupted by movements. Validation of such reduced-parameter studies or SSS solutions, using limited information to predict sleep profile, often indicate statistically significant success, when used on healthy people as many healthy people share similar sleep profiles that are significantly easier to guess right or correctly. However, as soon as the person deviates from the normal sleep profile, such as due to medication, clinical condition, or sleep disorders, these SSS methods become very inaccurate.

Examples of how medication, clinical conditions, or sleep disorders may impact the physiological signals being measured by a SSS may include, but are not limited to, one or more of the following examples.

First, in a case where a patient suffers from obstructive sleep apnea. Due to repeated breathing cessations associated with obstructive sleep apnea, blood oxygenation periodically drops, heart rate periodically rises and falls, and the patient may move or twitch in response to catching a large recovery breath after an apneic episode. These events often affect pulse, heart rate variability, and activity measured by a SSS device, causing these metrics to be different from what is observed in healthy people and negatively impacting the performance and accuracy of an automatic analysis of the impacted signals.

Second, in a case where a patient suffers from periodic limb movements. Due to this disorder the patient repeatedly suffers cortical arousals. Cortical arousals are short bursts of EEG activity indicating a period of wakefulness typically lasting only a few seconds. These cortical arousals may cause the patient to move and may cause the patients heart rate to surge and drop again. Furthermore, these cortical arousals may raise the patients blood pressure, causing a change in the measured PPG signal. Therefore, a SSS device measuring physiological signals other than the EEG signals may mis-classify the sleep state of the patient.

Third, in a case where a patient is taking medication, such as beta blockers for the treatment of high blood pressure, or where the patient has a pacemaker implanted to manage abnormal heart rhythms. Such a patient may not display the same heart rate variability and changes in heart rate variability as would a healthy person. And accordingly, a SSS device would very likely mis-classify the sleep state of the patient.

Fourth, in a case where a patient has an untreated heard condition causing abnormal heart rhythms, such as atrial fibrillation, supraventricular tachycardia, bradycardia, or heart failure. Such a patient may not display the same heart rate variability and changes in heart rate variability as would a healthy person, and a SSS device would very likely mis-classify the sleep state of the patient.

Fifth, in a case where a patient suffers from insomnia. Insomnia may cause frequent awakenings, cortical arousals, frequent activity, and heart rate variability. A SSS device measuring physiological signals other than the EEG signals may misclassify periods in the night where the patient has high heart rate variability and is lying awake but still as REM periods, despite the patient actually being awake. Furthermore, the SSS may overestimate sleep periods during times where the patient is lying still, relaxed, and drifting between sleep and wake.

Even though Simplified Sleep Studies (SSS) may not be able to determine accurately when the patient is sleeping or awake, the SSS device or system may be useful to record trends in the patients sleep. The inventors of this application have found that there is a significant need for SSS methods, devices, and systems that people would be willing to use frequently or every night for clinical purposes. And that if the accuracy and performance of such SSS could be improved, such SSS could be used to monitor sleep of those who's sleep deviates from a normal sleep profile, such as to determine the effectiveness of treatment for patients with sleep disorders, identifying if the person is trending to good or bad and adjust or adjust the treatment used, no matter if it is medications, therapy, or a medical device. And particularly, it is those patients outside the average, who's sleep profiles deviate from a normal sleep profile, who would benefit most from ready available and easily set up SSS. The inventors of this disclosure have found that to maximize the SSS usefulness, a personalized calibration of the SSS tailored to the specific patient using a High-Accuracy Sleep Study (HASS) on that same patient will allow a better determination of the sleep profile and tracking of the treatment effectiveness as the calibrated SSS output will more accurately reflect the sleep of the patient suffering, from a medical problem.

Personalized Sleep Classification (PSG)

The main goal of current SSS device manufacturers or providers is to provide sleep study solutions that work for most people in the general population, i.e., average people, or most common patient groups. Sometimes they can improve the performance of their studies to some degree by adding information about the person to the data used by the classifier, such as age, weight, gender, race/ethnicity and/or other factors that may affect the characteristics of the signals measured. However, the inventors of the present application have found that when it comes to sleep disorders and medication, the variables become so complicated that this type of profiling (i.e., adding information) will not work and does not provide accurate results or accurate sleep classification. Patients, people suffering from sleep disorders, and people on medication would however be the ones who would benefit the most from being continuously monitored during sleep.

As described below, to overcome this issue, the inventors of the present application have found that a personalized sleep classification can be achieved using the signals recorded and signal features derived from the signals. In various embodiments, this may include features such as pulse, heart rate, respiratory rate, pleth amplitude, pulse transit time or statistical features describing the signals such as amplitudes, averages, standard deviations, entropy, signal powerbands, signal coherence, signal correlation, cross spectral densities or power spectrum densities.

In a first embodiment, a method for creating a calibrated personalized sleep classification includes:
Obtaining recording of a simultaneous Simplified Sleep Studies (SSS) and Standard High-Accuracy Sleep Study (HASS);
Deriving signals and/or signal features from the SSS and having the HASS correctly scored;
Feeding the signals and/or signal features of the SSS to a classifier that predicts the sleep stages; and
Adjusting the classifier to “learn” to predict the outcome of the HASS, or reduce the uncertainty in the SSS, in the optimal way and thereby deriving a Personalized Sleep Classifier (PSC) that provides improved performance and/or accuracy based on the SSS signals.
Even further, this embodiment may further preferably include 660 further customizing the classifier by adding patient information to the classifier to improve the performance further. Examples of added patient information may include age, weight, gender, race/ethnicity, BMI, medication, and clinical conditions.

In a first embodiment, the High-Accuracy Sleep Study (HASS) may be a standard PSG study. In a preferred embodiment, each of the above steps, of obtaining of the recording of the simultaneous SSS and HASS; deriving signals and/signal features from the SSS and having the HASS correctly scored; feeding the signals and/or signal features of the SSS to the classifier that predicts the sleep stages; Adjusting the classifier to learn to predict the outcome of the HASS; and preferably customizing the classifier are done on a personalized basis for a specific, single subject or patient.

After the Personalized Sleep Classifier (PSC) has been derived, the classifier parameters take into account the specific characteristics of the subject that it was adapted to and is therefore not limited anymore to the outcome of general-population classifiers that are often used.

The signals and signal features from any previous night of SSS can be delivered as input to the PSC to check if the subject has been trending in any specific way and if the classifier parameters derived from the calibration nights are indeed typical for the sleep of the subject. This way the performance of the PSC for this particular person can be confirmed based on SSS signals obtained after the PSC has been derived and customized.

For any future night where the SSS signals are recorded, the PSC can be used for monitoring of the subjects sleep, or until the signals have drifted too far from the characteristics of the classifier parameters. In that case, a recalibration toward HASS study can be performed.

Calibration of Personalized Sleep Classification (PSC)

Even if the Personalized Sleep Classification (PSC) calibration can in theory be performed in large scale, it still requires a high-resolution sleep study (High-Accuracy Sleep Study (HASS)) to be performed, where the standard is often considered to be PSG or a Standard High-Accuracy Sleep Study (HASS). But with PSG being a complicated and expensive study, it would not be practical to have every subject that could benefit from PSC undergo a PSG. For this type of PSC calibration to become practical, a large-scale deployable high-resolution sleep study (HASS) is needed.

Along the sleep study spectrum of accuracy, performance, or precision, a standard HASS may be more accurate or may include more parameters than what may be termed a “practical” HASS. But a practical HASS may still provide higher accuracy when compared to, for example, a Simple Sleep Study (SSS). For example, a practical HASS may be based on a measure of even a single parameter, such as a respiratory or oximetry parameters, blood pressure parameters, or such as EEG, EOG, ECG and EMG signals, or a combination of such parameters. But even in this case, where the HASS is based on a single parameter or a combination of parameters, a practical HASS may stull provide a sufficiently accurate picture of the subjects sleep and sleep profile and capture the effects of sleep disorders.

Further, in another embodiment, a practical HASS method (as compared, for example, to a Standard HASS or PSG) may be based on a Self Applied Somnography (SAS). A SAS is a sleep study in which the sensors or sensing devices are configured such that the patient himself, as compared to an assistant or a certified or trained medical worker, may apply and monitor the performance of the sleep study sensors or sensing devices. SAS are designed to provide close to the same information and performance for sleep profiling as standard PSG or Standard HASS, or at least sufficiently accurate, and may even include a practical HASS. But SAS has the benefit that most people can successfully place the sensors on themselves and perform the recording. This allows the equipment and sensors to be delivered over the counter or by mail to the patient that can then perform the PSC calibration study himself before returning the SAS system to the clinic or shipping it back over mail.

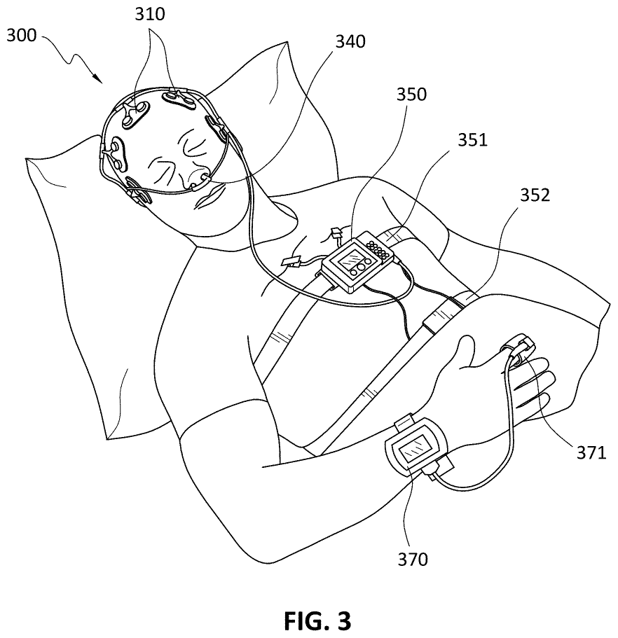

FIG. 3 shows a subject underling such a practical HASS according to this embodiment, in the form of a SAS. As shown in FIG. 3 a subject 300 may preferably apply the following sensors and devices to himself, or an untrained or uncertified assistant, such as a family member, roommate, or untrained or uncertified medical worker. EEG electrodes 310 may be attached to the head of the subject. The electrodes 310 may be arranged in a band to ensure proper placement. The band may also include, but does not necessarily include, EOG electrodes 320 placed on one or more distal ends of the headband so as to be arranged near an eye of the subject 300. Furthermore, the patient 300 may have a nasal cannula 340 used to measure nasal breathing. The subject 300 may also have respiratory inductance plethysmography (RIP) streatchable belts 351, 352 placed around his chest (thoracic) and abdomen, respectively, to measure breathing movements. Stretchable belts 351,352 may contain a conductor (not shown) that when put on a subject 300, form a conductive loop that creates an inductance that is directly proportional to the absolute cross sectional area of the body part that is encircled by the loop. When such a belt is placed around the abdomen or thorax, the cross-sectional area is modulated with the respiratory movements and therefore also the inductance of the belt. Conductors in the belts may be connected to signal processor 350 by leads or transmitted or received by the processor 350 wirelessly. Processor 350 may include a memory storage. By measuring the belt inductance, a value is obtained that is modulated directly proportional with the respiratory movements. RIP technology includes therefore an inductance measurement of conductive belts that encircle the thorax and abdomen of a subject.

The subject 300 of the embodiment of FIG. 3 may also have a pulse oximeter 370 on the wrist and a corresponding sensor 371 on a finger, such as an index finger, to measure the blood oxygen saturation and pulse in the finger. Furthermore, the patient may also have leg EMG leads although not shown in FIG. 3.

In another embodiment, an advanced SAS system may be used that is based on the use of wireless Smart Sensors that minimize the use of cables and further simplify significantly the hookup of the sensors while maintaining the performance and high-resolution of the cabled SAS or PSG methods. Such an advanced SAS is also preferable in that movement or turning of the subject during the study is not encumbered by wires or cables.

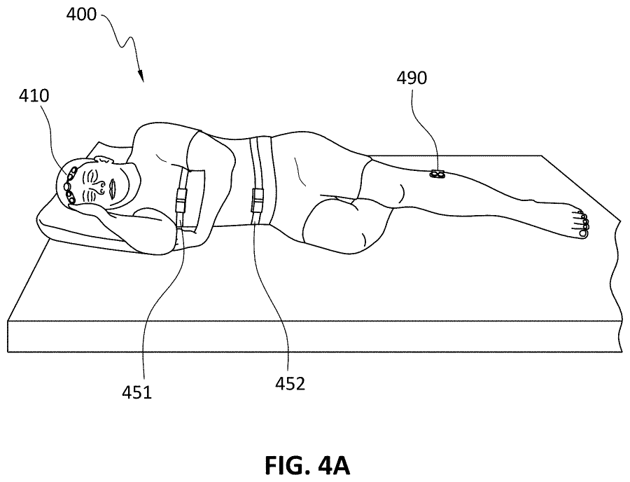

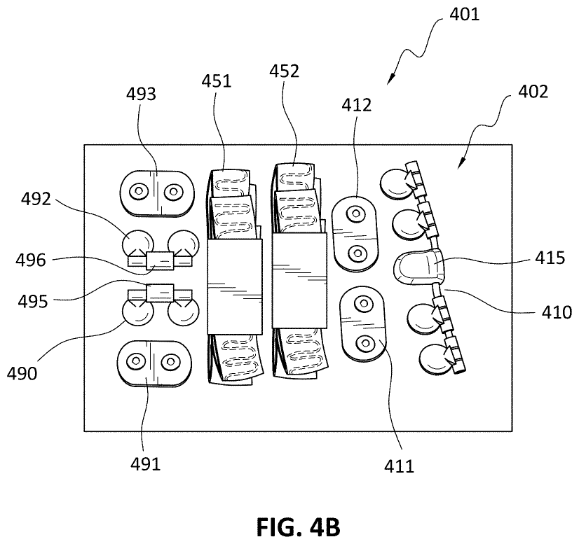

FIG. 4A shows a schematic of a subject 400 sleeping with the wireless SAS sleep study. The devices worn by the subject in such as study may include EEG electrodes 410 placed on the forehead of the subject, respiratory inductance plethysmography (RIP) belts 451, 452, and a sensor or leg EMG lead 490 applied to the leg of the subject. As noted above, these sensors and devices have been provided to the subject in such a way that the subject can easily and consistently apply the sensors and devices himself (or herself).

FIG. 4B shows a kit 401 of sleep sensors that may be provided to the subject, for example, over the counter or by mail contained within an envelope or packaging 402. The kit 401 may include EEG electrodes 410 with respectively contacts and electrodes 411 and 412 to be placed on the scalp of the subject, for example on the forehead of the subject. A signal obtaining/transmitting device 415 is arranged to receive signals obtained from contacts and electrodes 411, 412 either through wires or wirelessly. The kit 401 also includes respiratory inductance plethysmography (RIP) belts 451, 452, and may also include a RIP signal processor (not shown). Additional sensors may also be included in the kit, including one or more leg EMG leads 490, 492 with corresponding contacts and electrodes 491,493 and corresponding signal obtaining/transmitting devices 495,496 to be applied to a leg of the subject.

The kit 401 shown in FIG. 4B may be used as a practical, wireless SAS HASS sleep study. Further, the kit 401 may be used as a SSS study which is calibrated by an even higher accuracy HASS study.

For maximum usability of the output of PSC, it is important to know its performance. This can be done by comparing the predictions of the PSC to the results from one or more HASS studies. Furthermore, the performance can be estimated by investigating trends and variance in the PSC outcomes without comparing them to results of a HASS.

Method for Personalized Sleep Classifier (PSC) Calibration

Normally it is necessary to have large training datasets available for creating sleep classifiers. Such sleep classifiers are therefore normally not made for a specific person but use hundreds or thousands of recorded nights of sleep from multiple persons. A general sleep classifier is therefore designed to fit all persons, average persons, or a specific group of persons described by the training set. A general classifier can be developed to perform well on average when predicting sleep profiles compared to the results of a HASS as the variance of the sleep profile within the group may not be large on average. But as described above, such general sleep classifiers become extremely inaccurate when applied to subjects who are outside the average, who's sleep profiles deviate from a normal sleep profile. Further, can be very difficult to train a sleep classifier for a single person as that would require a lot of reference nights recorded. To avoid this issue, a general classifier can be used as the basis for the creation of a Personalized Sleep Classifier (PSC). The classifier parameters of the general classifier are adjusted after the HASS to increase the performance for the individual person that the PSC is intended for. This adjustment can be achieved by using statistical methods that adjust the outputs of the general classifier to fit to the individual.

According to different embodiments, several methods can be applied to adjust a general classifier (base classifier) to become a PSC for an individual person, including but not limited to one or more, of a combination of: scaling, such as Platt scaling or Isotonic scaling or regression, or statistical scaling; normalizations; training or retraining a general base classifier; using known personal information; and/or increasing the classifier parameter training dataset, as described in more detail below.

In one embodiment, Platt scaling, Isotonic regression, or similar methods may be used to adjust a general classifier (base classifier) to become a PSC for an individual person. In these methods, the outputs of a base classifier are calibrated such that they represent the probability of a correct classification for the individual person or increase the performance of the classifier.

In another or the same embodiment, normalization of input data to a base classifier may be used to adjust the general classifier (base classifier) to become a PSC for an individual person. In these methods, the normalization of the input data is customized to the individual person that the PSC is intended for.

In another or the same embodiment, training or retraining of a general base classifier may be used to adjust a general classifier (base classifier) to become a PSC for an individual person. For example, the last or a few last layers of an artificial neural network may be used, where the neural network output is customized to the individual person.

In another or the same embodiment, the individual person's personal information, such as age, weight, gender, race/ethnicity, BMI, medication and health condition may be used as input to further improve selection of the base classifier.

In another or the same embodiment, increasing the classifier parameter training dataset and PSC performance may be performed to adjust a general classifier (base classifier) to become a PSC for an individual person. These may include using the person's information to locate data from other persons with similar profiles and thereby create a group for training the classifier. This may also include suing the group data to adjust a base classifier to the group's HASS and thereby become a group-specific classifier. And this may also include further adapting the group-specific classifier to increase the performance for the target person and derive the PSC.

Another embodiment may include calibrating an automatic classifier, including but not limited to a Nox BodySleep classifier in the medical software application Noxturnal 6.2. (Noxturnal is used here as a non-limiting example of a software system provided by Nox Medical that provides automatic analysis, scoring, and advanced reporting tools for sleep studies.) The Noxturnal software system uses an activity signal obtained by an activity sensor, and the chest and abdomen respiratory inductance plethysmography (RIP) signals to estimate wake, REM sleep and non-REM sleep. In this embodiment the calibration would be conducted by performing a PSG or a SAS HASS sleep study at the same time as measuring the activity and RIP signals used by the Nox BodySleep classifier. In this embodiment the calibration could be performed by applying Platt scaling or Isotonic scaling or regression to transform the outputs of the Nox BodySleep classifier to change the decision boundaries and customize the output of the Nox BodySleep classifier to the individual.

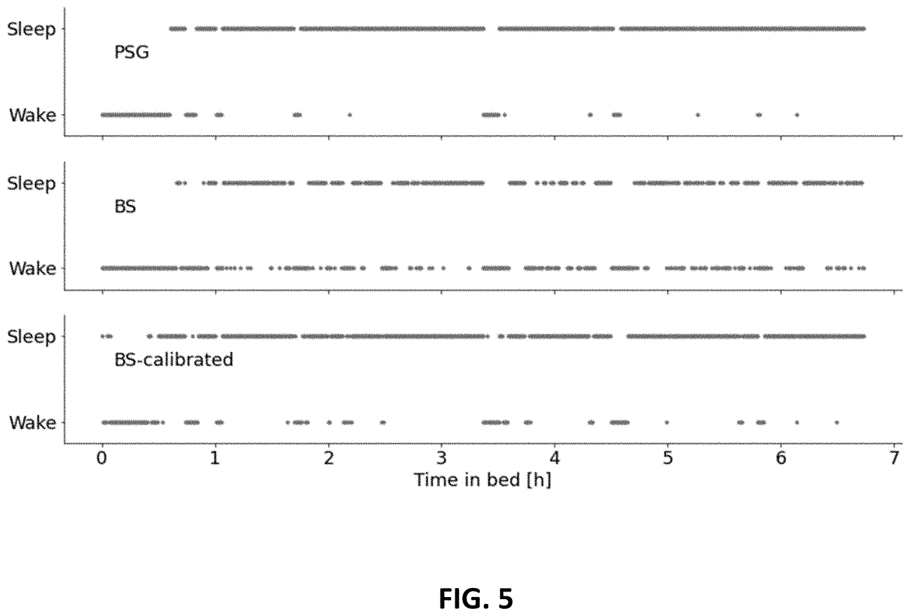

In the above-mentioned embodiment sleep data from a HASS sleep study can be used to calibrate a SSS sleep study classifier when the two sleep studies, HASS and SSS, are performed simultaneously on the individual subject. The calibrated SSS sleep study classifier can subsequently have higher performance and accuracy than the non-calibrated SSS classifier. That is, the calibrated SSS sleep study classifier better approaches, aligns with, or fits the sleep profiled obtained by the PSG. FIG. 5 shows an example of how the calibration improves the classification of wake and sleep periods. FIG. 5 shows the classification of wake and sleep in each 30 second period in in an overnight HASS (PSG in this case) sleep study, which is labelled PSG in FIG. 5. The sleep/wake classification of the PSG sleep study in FIG. 5 is plotted as a reference to gauge the performance of the Nox BodySleep (BS) SSS classifier before and after calibration using Platt scaling. The calibration was done by simultaneously recording two nights of the PSG HASS sleep study and the Nox BodySleep (BS) SSS sleep study and using the data from the first night to calibrate the Nox BodySleep SSS sleep/wake classifier. The data in FIG. 5 shows how the calibration improves the performance of the Nox BodySleep (BS) SSS sleep/wake classifier when applied to the second sleep recording (BS-calibrated).

The sleep/wake classification of the Nox BodySleep classifier is labelled BS in FIG. 5. The sleep/wake classification of the Nox BodySleep (BS) classifier shows considerably more wake events than the sleep/wake classification of the PSG sleep study. The sleep/wake classification of the calibrated Nox BodySleep is labelled as BS-calibrated in FIG. 5. FIG. 5 shows that there is considerably higher agreement between the PSG sleep/wake classification and the calibrated Nox BodySleep sleep/wake classification (BS-calibrated), than the agreement between the PSG sleep/wake classification and the non-calibrated Nox BodySleep sleep/wake classification (BS). By performing the calibration the agreement of the sleep/wake classification between the PSG and Nox BodySleep was improved from 55% to 78% between the PSG and the calibrated Nox BodySleep classifier.

Another embodiment includes calibrating an automatic classifier, including but not limited to the Nox BodySleep classifier in the medical software application Noxturnal 6.2, which uses an activity signal obtained by an activity sensor, and chest and abdomen respiratory inductance plethysmography (RIP) signals obtained from chest and abdomen RIP belts to estimate wake, REM sleep and non-REM sleep. In this embodiment data from several nights of simultaneous sleep recordings using a PSG or SAS HASS sleep study at the same time as measuring the activity and RIP signals used by the Nox BodySleep classifier. In this embodiment the last few layers of the Nox BodySleep classifier could be retrained using the simultaneous data to calibrate the outputs of the Nox BodySleep to the individual.

Another embodiment of the method could be to calibrate an automatic classifier, including but not limited to the Nox BodySleep classifier in the medical software application Noxturnal 6.2, which uses an activity signal obtained by an activity sensor, and the chest and abdomen respiratory inductance plethysmography (RIP) signals to estimate wake, REM sleep and non-REM sleep. In this embodiment HASS and SSS data from a focused group of patients sharing the same characteristics such as age, sex, and clinical condition are used to create a SSS classifier customized to the group using methods as described above. The resulting PSC then better represents the group of individuals.

Another embodiment of the method could be to calibrate an automatic classifier, including but not limited to the Nox BodySleep classifier in the medical software application Noxturnal 6.2, which uses an activity signal obtained by an activity sensor, and the chest and abdomen respiratory inductance plethysmography (RIP) signals to estimate wake, REM sleep and non-REM sleep. In this embodiment patient data, including but not limited to age, sex, race, body mass index (BMI), medical condition, or a physiological signal, is used to create an embedding space of the individual or a group of patients. This embedding space can then be used to correct for patient characteristics and use a more generalized SSS classifier to get the customized result. In this embodiment the embedding space could include but would not be limited to sex with a binary variable of 1 for the patient with an untreated medical condition and −1 for the patient under treatment. By using the embedding space the classifier output could be calibrated to take into account the treatment effects or not.

In the above mentioned embodiments of SSS, some examples have been provided that include Nox BodySleep classifier and Noxturnal. But these embodiments are provided here as a non-limiting example of a software system or sensors. Other sleep study or sleep analysis software or programs could also similarly be used, such as any actigraphy, a smart watch, a pulse sensor, a pulse oximeter, a wearable Peripheral Arterial Tone (PAT) device, a smart bed mattress such as a sleep mattress that picks up heartbeat and breathing from the mattresses pressure, a sensor strip placed under or around the patient, a radar based sensor system, or any other system performing a measurement of the patient or their surroundings for the intention of monitoring sleep or sleep events or combination thereof. These may further include Apple's Sleep ap in watchOS 7 or Apple's Health app or Apple's Bedtime feature on iOS, or any other sleep analysis software, apps or systems such as those provided by Beddit, Fitbit, Oura, or ActiWatch. Further, although some embodiments of the SSS described herein are based on or include RIP belts, the SSS is not limited to such sensors, and may include a different parameter or other parameters or sensors, such as actigraphy, accelerometers, one or more respiratory sensors, pulse, heart rate, respiratory rate, pleth amplitude, pulse transit time, eye movement, sweat rates or skin capacitance, electrodermal activity, skin conductance, other parameters measured in PSG including one or more of a combination of electroencephalography (EEG), electrocardiography (ECG), electrooculography (EOG), and electromyography (EMG), or statistical features describing such signals such as amplitudes, averages, standard deviations, entropy, signal powerbands, signal coherence, signal correlation, cross spectral densities or power spectrum densities.

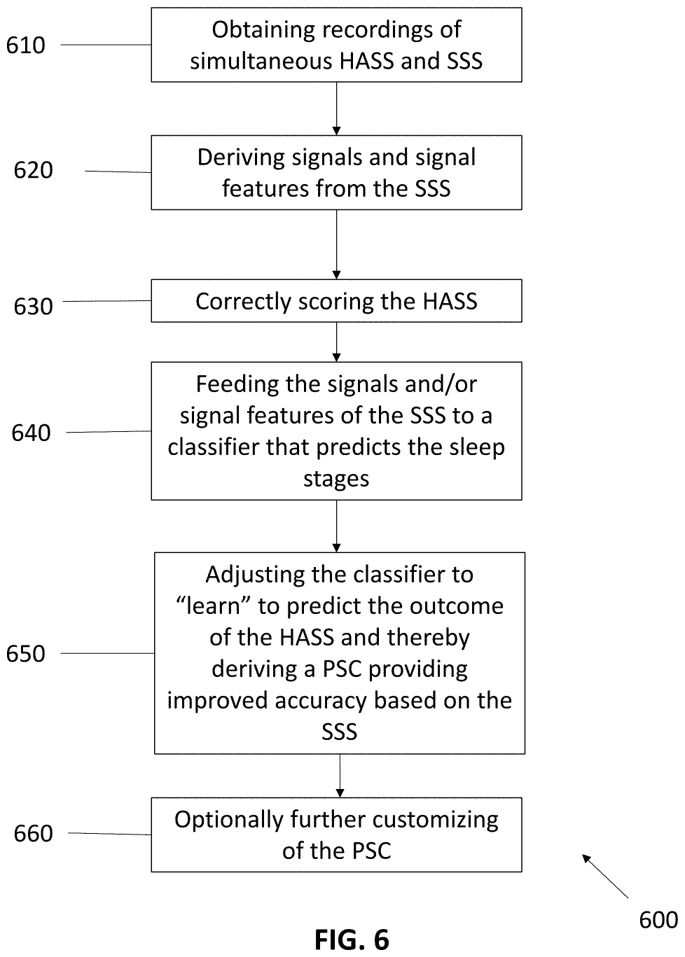

FIG. 6 shows an embodiment of a method 600 for creating a calibrated personalized sleep classification includes: 610 Obtaining recording of a simultaneous Simplified Sleep Studies (SSS) and Standard High-Accuracy Sleep Study (HASS); 620 Deriving signals and/or signal features from the SSS and 630 having the HASS correctly scored; 640 Feeding the signals and/or signal features of the SSS to a classifier that predicts the sleep stages; and 650 adjusting the classifier to “learn” to predict the outcome of the HASS, or reduce the uncertainty in the SSS, in the optimal way and thereby deriving a Personalized Sleep Classifier (PSC) that provides improved performance and/or accuracy based on the SSS signals. Even further, this embodiment may further preferably include 660 further customizing the classifier by adding patient information to the classifier to improve the performance further. Examples of added patient information may include age, weight, gender, race/ethnicity, BMI, medication, and clinical conditions.

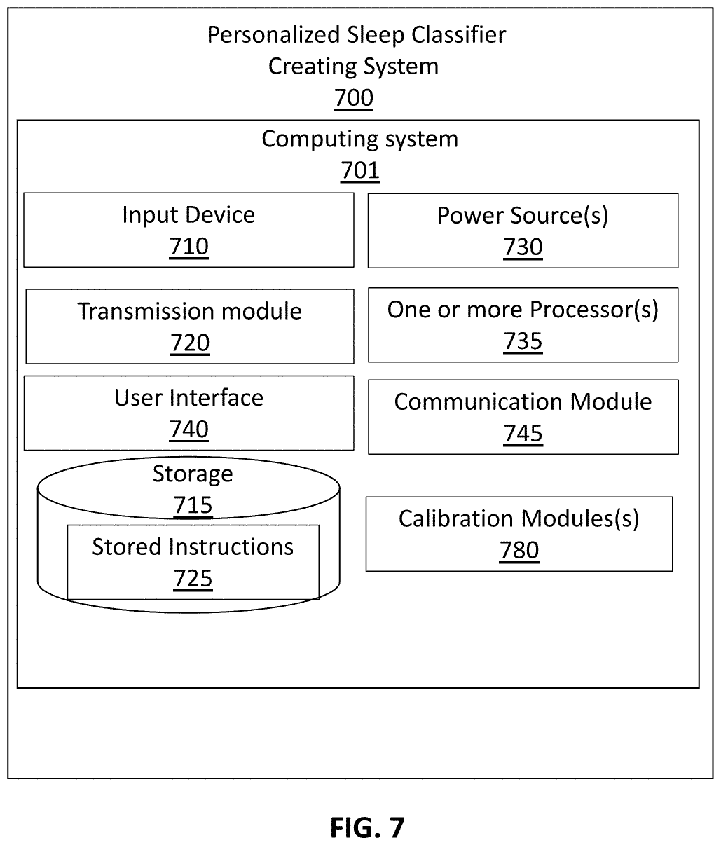

FIG. 7 shows personalized sleep classifier creating system 700 according to an embodiment, or in other words a system that creates a personalized sleep classifier for a subject. The system 700 includes a computing system. The computing system 701 may include and use a special-purpose or a general-purpose computer system that includes computer hardware, such as, for example, one or more processors 735 and system memory or storage 715. Storage 715 may have stored instructions 725 stored thereon, which, when executed by the one or more processors 735 cause the one or more processors 735 to perform a method for creating a personalized sleep classifier, for example, to perform the method shown in FIG. 6. Computer system 701 may include an input device 710, which is configured to receive input either with hardware wires or through wireless connections. For example, the input device 710 may be configured to receive sleep data from biosignals received from a subject of a High-Accuracy Sleep Study (HASS) and sleep data from biosignals received from the subject in a Simplified Sleep Study (SSS). Such sleep data may be retrieved as pre-recorded data from a memory storage and received by the input device 710 across a local network, from a local database, from a remote database, and over the Internet through an Internet connection. Alternatively, the computer system 701 may receive sleep data in real-type or delayed time directly from sleep sensors or one or more data processors, such as the devices shown in the embodiments of FIG. 1, FIG. 3, and FIGS. 4A and 4B, again through either direct wiring or wireless communication. Transmission module 720 may communicate with devices outside the computer system, such as to sleep data sensors or sleep data processors, such as those shown in the embodiments of FIG. 1, FIG. 3, and FIGS. 4A and 4B, through either a wire connection of wireless communication, in a local network or across a data connection. The computer system 701 may include a user interface, which may include a display, for example, a display such as those of FIGS. 2A and 2B or of FIG. 5. The user interface may further include devices to receive input from a user, such as a keyboard or mouse, or touchscreen. Computing system 701 according to the embodiment further includes power source 730, a communication module 745, and calibration module 780, used in the calibration of the personalized sleep classifier. Calibration module 780 may include an artificial intelligence module or an AI engine to perform the calibration of the personalized sleep classifier as described herein. It is noted that computing system 701 is not necessarily arranged locally to the subject for which the personalized sleep classifier is being created. Rather, the computer system 701 may received from the sleep data from biosignals received from the subject of the High-Accuracy Sleep Study (HASS) and sleep data from biosignals received from the subject in the Simplified Sleep Study (SSS) as was previously recorded and sent, for example, by the subject by mail or across a data connection.

Although the subject matter of this disclosure has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

Embodiments of the present disclosure, including the development or creation of the classifiers and or calibration or recalibration of the classifiers, may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures, for example, for the development or creation of the classifiers and or calibration or recalibration of the classifiers. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

Certain terms are used throughout the description and claims to refer to particular methods, features, or components. As those having ordinary skill in the art will appreciate, different persons may refer to the same methods, features, or components by different names. This disclosure does not intend to distinguish between methods, features, or components that differ in name but not function. The figures are not necessarily drawn to scale. Certain features and components herein may be shown in exaggerated scale or in somewhat schematic form and some details of conventional elements may not be shown or described in interest of clarity and conciseness.

Although various example embodiments have been described in detail herein, those skilled in the art will readily appreciate in view of the present disclosure that many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

1. A method for creating a personalized sleep classifier for a subject, the method comprising:
obtaining sleep data from biosignals received from the subject in a High-Accuracy Sleep Study (HASS) and sleep data from biosignals received from the subject in a Simplified Sleep Study (SSS), the data from the High-Accuracy Sleep Study (HASS) being obtained from the subject during a period of time that is simultaneous with the period during which the data from the Simplified Sleep Study (SSS) is obtained from the subject;
developing a high-resolution HASS sleep profile from the sleep data of the High-Accuracy Sleep Study (HASS);
creating a personalized sleep classifier that outputs a SSS sleep profile of the subject based on the sleep data from the Simplified Sleep Study (SSS);
calibrating the personalized sleep classifier such that the SSS sleep profile output by the personalized sleep classifier based on the Simplified Sleep Study (SSS) of the subject approaches or aligns with the high-resolution HASS sleep profile based on the High-Accuracy Sleep Study (HASS) of the subject.

2. The method according to claim 1, wherein obtaining the sleep data from the High-Accuracy Sleep Study (HASS) and the sleep data from the Simplified Sleep Study (SSS) includes both performing the High-Accuracy Sleep Study (HASS) on the subject and simultaneously performing the Simplified Sleep Study (SSS) on the subject.

3. The method according to claim 1, wherein obtaining the sleep data from the High-Accuracy Sleep Study (HASS) and the sleep data from the Simplified Sleep Study (SSS) includes retrieving the sleep data from the High-Accuracy Sleep Study (HASS) or the sleep data from the Simplified Sleep Study (SSS) as pre-recorded data from a memory storage.

4. The method according to claim 1, wherein the biosignals received from the subject in the High-Accuracy Sleep Study (HASS) include Electroencephalography (EEG), Electrooculography (EOG), Electromyography (EMG), signals obtained from a nasal cannula, thoracic and/or abdomen, or pulse oximetry signals.

5. The method according to claim 1, wherein the biosignals received from the subject in the Simplified Sleep Study (SSS) include one or more of a thoracic RIP signal, an abdomen RIP signal, a pulse signal, an activity signal, or an oximetry signal.

6. The method according to claim 1, further comprising determining an accuracy of the personalized sleep classifier by running the personalized sleep classifier on previous nights of sleep recorded in the Simplified Sleep Study (SSS) and determining the variance of the SSS sleep profile and the HASS sleep profile for the same previous nights of sleep.

7. The method according to claim 1, wherein the High-Accuracy Sleep Study (HASS) is a standard polysomnography (PSG).

8. The method according to claim 1, wherein the High-Accuracy Sleep Study (HASS) is a Self Applied Somnography (SAS).

9. The method according to claim 1, wherein calibrating the personalized sleep classifier include one or more of statisitical scaling, Platt scaling, or isotonic regression or scaling of input data to the personlized classifier; statisitical scaling, Platt scaling, or isotonic regression or scaling of an output of the personlized sleep classifier; training or retraining at least a part of the personalized sleep classifier; normalization of input data to the personlized classifier; using known personal information of the subject; increasing the classifier parameter training dataset.

10. A method for identifying sleep stages or sleep events of the subject, or providing a sleep profile for a subject comprising;
creating a personalized sleep classifier for a subject according to claim 1; and
identifying sleep stages or sleep events of the subject, or providing a sleep profile of the subject, based on further sleep data from the subject from a further Simplified Sleep Study (SSS) using the personalized sleep classifier,
wherein the further sleep data is obtained from further Simplified Sleep Study (SSS) of a different period of time, either before or after, the sleep data from the biosignals received from the subject in the Simplified Sleep Study (SSS) were recorded or obtained.

11. A method for diagnosing a sleep disorder of a subject comprising:
creating a personalized sleep classifier for a subject according to claim 1; and
diagnosing the sleep disorder by identifying sleep stages or sleep events of the subject, or providing a sleep profile of the subject, based on further sleep data from the subject from a further Simplified Sleep Study (SSS) using the personalized sleep classifier,
wherein the further sleep data is obtained from further Simplified Sleep Study (SSS) of a different period of time, either before or after, the sleep data from the biosignals received from the subject in the Simplified Sleep Study (SSS) were recorded or obtained.

12. A method for determining an efficacy of a treatment of a subject comprising: creating a personalized sleep classifier for a subject according to claim 1; and
determining the efficacy of a sleep treatment by identifying sleep stages or sleep events of the subject, or providing a sleep profile of the subject, based on further sleep data from the subject from a further Simplified Sleep Study (SSS) using the personalized sleep classifier,
wherein the further sleep data is obtained from further Simplified Sleep Study (SSS) of a different period of time, either before or after, the sleep data from the biosignals received from the subject in the Simplified Sleep Study (SSS) were recorded or obtained.

13. A method for identifying sleep stages of a subject comprising;
creating a personalized sleep classifier for a subject according to claim 1; and
using chest and abdomen respiratory inductance plethysmography (RIP) signals obtained in a subsequent Simplified Sleep Study (SSS) to estimate wake, REM sleep and non-REM sleep stages in the subject.

14. A hardware storage device having stored thereon computer executable instructions which, when executed by one or more processors of a computer system, configure the computer system to perform the method according to claim 1.

15. A method for creating a personalized sleep classifier for one or more subjects of a focused group of subjects, the method comprising:
obtaining sleep data from biosignals received from the focused group of subjects in a High-Accuracy Sleep Study (HASS) and sleep data from biosignals received from the focused group of subjects in a Simplified Sleep Study (SSS), the data from the High-Accuracy Sleep Study (HASS) being obtained from the focused group of subjects during a period of time that is simultaneous with the period during which the data from the Simplified Sleep Study (SSS) is obtained from the focused group of subjects;
developing a high-resolution HASS sleep profile from the sleep data of the High-Accuracy Sleep Study (HASS);
creating a personalized sleep classifier that outputs a SSS sleep profile of the focused group of subjects based on the sleep data from the Simplified Sleep Study (SSS);
calibrating the personalized sleep classifier for one or more of the focused group of subjects such that the SSS sleep profile output by the personalized sleep classifier based on the Simplified Sleep Study (SSS) of the one or more of the focused group of subjects approaches or aligns with the high-resolution HASS sleep profile based on the High-Accuracy Sleep Study (HASS) one or more of the focused group of subjects,
wherein the focused group of subjects share one or more same characteristics, including age, sex, diagnosed clinical condition, weight, race/ethnicity, BMI, treatment with a same medication, or health condition.

16. A method for identifying sleep stages or sleep events of the subject, or providing a sleep profile for a subject comprising;
creating a personalized sleep classifier for a subject according to claim 15; and
identifying sleep stages or sleep events of the subject, or providing a sleep profile of the subject, based on further sleep data from the subject from a further Simplified Sleep Study (SSS) using the personalized sleep classifier,
wherein the further sleep data is obtained from further Simplified Sleep Study (SSS) of a different period of time, either before or after, the sleep data from the biosignals received from the subject in the Simplified Sleep Study (SSS) were recorded or obtained.

17. A computing system for creating a personalized sleep classifier, the computing system comprising:
one or more processors;
one or more computer-readable storage devices having stored thereon computer-executable instructions that are structured such that, when executed by the one or more processors, cause the computing system to perform the following:
obtain sleep data from biosignals received from the subject of a High-Accuracy Sleep Study (HASS) and sleep data from biosignals received from the subject in a Simplified Sleep Study (SSS), the data from the High-Accuracy Sleep Study (HASS) being obtained from the subject during a period of time that is simultaneous with the period during which the data from the Simplified Sleep Study (SSS) is obtained from the subject;
develop a high-resolution HASS sleep profile from the sleep data of the High-Accuracy Sleep Study (HASS);
create a personalized sleep classifier that outputs a SSS sleep profile of the subject based on the sleep data from the Simplified Sleep Study (SSS);
calibrate the personalized sleep classifier such the SSS sleep profile output by the personalized sleep classifier based on the Simplified Sleep Study (SSS) of the subject approaches or aligns with the high-resolution HASS sleep profile based on the High-Accuracy Sleep Study (HASS) of the subject.

18. The computing system according to claim 17, further comprising a storage device that stores the data from the High-Accuracy Sleep Study (HASS) and the data from the Simplified Sleep Study (SSS).

19. The computing system according to claim 17, further comprising a receiver configured to receive as input the data from the High-Accuracy Sleep Study (HASS) and the data from the Simplified Sleep Study (SSS).

20. The computing system according to claim 17, wherein the one or more computer-readable storage devices further have stored thereon computer-executable instructions that are structured such that, when executed by the one or more processors, cause the computing system to
identify sleep stages of the subject based on further sleep data from subsequent biosignals received from the subject in a subsequent Simplified Sleep Study (SSS) using the personalized sleep classifier.
